(19) United States
(12) Patent Application Publication
(10) Pub. No.: US 2025/0290900 A1
(43) Pub. Date: Sep. 18, 2025

(54) ULTRASONIC DIAGNOSTIC APPARATUS

(71) Applicant: FUJIFILM Corporation, Tokyo (JP)

(72) Inventors: Taizo YAMAWAKI, Tokyo (JP); Takanori HIRAI, Tokyo (JP); Tsuneo KAWAMATA, Tokyo (JP); Koki YAMAGUCHI, Tokyo (JP)

(73) Assignee: FUJIFILM Corporation, Tokyo (JP)

(21) Appl. No.: 19/074,822

(22) Filed: Mar. 10, 2025

(30) Foreign Application Priority Data
Mar. 15, 2024 (JP) 2024-041119

(51) Int. Cl. G01N 29/06; G01B 7/008

(52) U.S. Cl. CPC G01N 29/0654; G01B 7/008; G01N 2291/023

(57) ABSTRACT

An ultrasound diagnostic apparatus includes an ultrasound probe, a position sensor, and an information processing unit. The information processing unit includes a position measurement unit, an ultrasound transmitter and receiver, an image generation unit, and a controller. The information processing unit generates ultrasound image data on the basis of ultrasound waves received by the ultrasound probe and measure a position of the ultrasound probe on the basis of a sensor signal output from the position sensor. The ultrasound diagnostic apparatus further includes a control database in which a frame rate in a case where the ultrasound image data is generated and a measurement frequency of the sensor signal are associated with each other, and the information processing unit sets at least one of the frame rate or the measurement frequency on the basis of the control database.

### CROSS REFERENCE TO RELATED APPLICATION

This application claims priority to Japanese Patent Application No. 2024-041119 filed on Mar. 15, 2024 which is incorporated herein by reference in its entirety including the specification, claims, drawings, and abstract.

### BACKGROUND OF THE INVENTION

### 1. Field of the Invention

The present disclosure relates to an ultrasound diagnostic apparatus, and particularly to position measurement of an ultrasound probe.

### 2. Description of the Related Art

There is an ultrasound diagnostic apparatus in which a position sensor is provided in an ultrasound probe, and the position of the ultrasound probe in a case of acquiring an ultrasound image is measured. WO2014/156973A discloses such an ultrasound diagnostic apparatus. JP1994-347527A (JP-H06-347527A) discloses a position sensor that detects an electromagnetic field.

### SUMMARY OF THE INVENTION

In general, in an ultrasound diagnostic apparatus that measures the position of an ultrasound probe, a position sensor and a vibration element of the ultrasound probe are disposed close to each other. Therefore, in a case of acquiring an ultrasound image, transmission signals applied to the vibration element may interfere with sensor signals output from the position sensor, and an error may occur in the measurement position of the ultrasound probe.

An object of the present disclosure is to improve accuracy of position measurement in an ultrasound diagnostic apparatus that measures a position at which an ultrasound image is acquired.

An ultrasound diagnostic apparatus according to an aspect of the present disclosure is an ultrasound diagnostic apparatus including an ultrasound probe, and a position sensor that is provided in the ultrasound probe, the ultrasound diagnostic apparatus including an information processing unit configured to generate ultrasound image data on the basis of ultrasound waves received by the ultrasound probe and measure a position of the ultrasound probe on the basis of a sensor signal output from the position sensor; and a control database in which a frame rate in a case where the ultrasound image data is generated and a measurement frequency of the sensor signal are associated with each other, in which the information processing unit sets at least one of the frame rate or the measurement frequency on the basis of the control database.

In one embodiment, the control database includes a measurement frequency table in which the frame rate and an interference-avoidance measurement frequency are associated with each other, and the information processing unit sets the measurement frequency to the interference-avoidance measurement frequency associated with the frame rate in the measurement frequency table.

In one embodiment, the information processing unit corrects a position measurement value obtained by the sensor signal, on the basis of an offset adjustment value obtained in advance, updates, in a case where the measurement frequency is changed, the offset adjustment value on the basis of a difference between the corrected position measurement value obtained before the measurement frequency is changed and the position measurement value before correction obtained after the measurement frequency is changed, and corrects the position measurement value before correction obtained after the measurement frequency is changed, on the basis of the offset adjustment value before the update.

In one embodiment, the control database includes a frame rate table in which the measurement frequency and an interference-avoidance frame rate are associated with each other, and the information processing unit sets the frame rate to the interference-avoidance frame rate associated with the measurement frequency in the frame rate table.

In one embodiment, the information processing unit changes the number of ultrasound pulses transmitted by the ultrasound probe per single transmission period to change the frame rate.

In one embodiment, the information processing unit causes, among a plurality of ultrasound pulses transmitted by the ultrasound probe per single transmission period, the ultrasound pulses increased compared to before the frame rate is changed not to contribute to generation of the ultrasound image data.

According to the present disclosure, it is possible to improve accuracy of position measurement in an ultrasound diagnostic apparatus that measures a position at which an ultrasound image is acquired.

### BRIEF DESCRIPTION OF THE DRAWINGS

FIG. 1 is a diagram illustrating a configuration of an ultrasound diagnostic apparatus according to an embodiment of the present disclosure.

FIG. 2 is a diagram illustrating a time waveform of a transmission signal and a sensor signal.

FIGS. 3A and 3B are diagrams illustrating variation in position coordinate values with respect to a measurement frequency for two different frame rates.

FIG. 4 is a diagram illustrating transmission signals before and after a frame rate is changed.

FIG. 5 is a diagram illustrating transmission signals before and after a frame rate is changed.

FIG. 6 is a diagram illustrating an example of an offset error with respect to a measurement frequency.

FIG. 7 is a timing chart of processing of changing an offset adjustment value in accordance with a change in measurement frequency.

### DESCRIPTION OF THE PREFERRED EMBODIMENTS

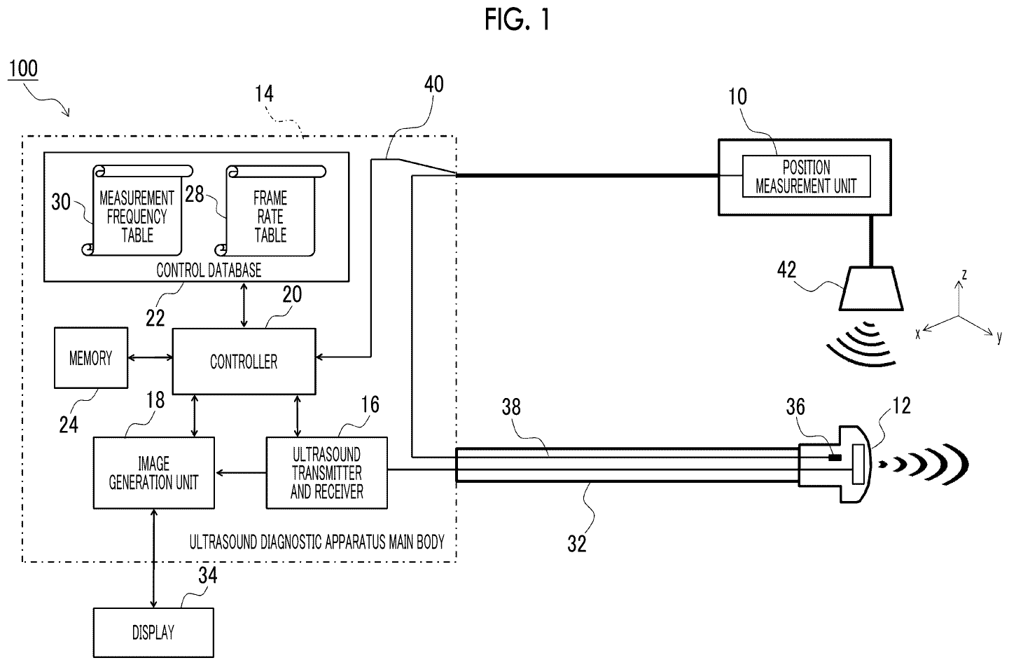

FIG. 1 is a diagram illustrating a configuration of an ultrasound diagnostic apparatus 100 according to an embodiment of the present disclosure. The ultrasound diagnostic apparatus 100 includes a position measurement unit 10, a position measurement transmitter 42, an ultrasound probe 12, a position sensor 36, an ultrasound diagnostic apparatus main body 14, and a display 34. The ultrasound diagnostic apparatus main body 14 includes an ultrasound transmitter and receiver 16, an image generation unit 18, a controller 20, a control database 22, and a memory 24. The ultrasound probe 12 is connected to the ultrasound transmitter and receiver 16 via a cable 32.

The position measurement unit 10, the ultrasound transmitter and receiver 16, the image generation unit 18, and the controller 20 constitute an information processing unit as hardware.

The information processing unit may include a processor that executes a program to configure a part or all of the components (the position measurement unit 10, the ultrasound transmitter and receiver 16, the image generation unit 18, and the controller 20). In addition, the information processing unit may include an electronic circuit as an interface for the ultrasound probe 12, the memory 24, and the control database 22. The information processing unit is configured to generate ultrasound image data on the basis of the ultrasound waves received by the ultrasound probe 12 and measure the position of the ultrasound probe 12 on the basis of the sensor signal output from the position sensor 36. The control database 22 may be information stored in a recording medium that is hardware. The control database 22 may be configured in the memory 24.

The controller 20 performs overall control of the ultrasound diagnostic apparatus 100. The ultrasound transmitter and receiver 16 outputs a transmission signal to the ultrasound probe 12 to cause the ultrasound probe 12 to transmit an ultrasonic wave. The ultrasonic waves transmitted from the ultrasound probe 12 form an ultrasonic beam directed in a specific direction, and the ultrasonic beam is scanned across a subject. The ultrasound probe 12 receives the ultrasound waves reflected by the subject in each direction in which the ultrasound beam is directed. The ultrasound probe 12 converts ultrasonic waves into electric signals and outputs the electric signals to the ultrasound transmitter and receiver 16. The ultrasound transmitter and receiver 16 performs processing such as detection and amplification on the electric signals as reception signals output from the ultrasound probe 12. In addition, the ultrasound transmitter and receiver 16 performs phase summation of the reception signals obtained from each direction where the ultrasound beam is directed, and outputs the reception signals after phase summation (hereinafter, referred to as phase summation reception signal) to the image generation unit 18.

The image generation unit 18 generates ultrasound image data, such as B-mode image data, on the basis of the phase summation reception signals output from the ultrasound transmitter and receiver 16 for each direction in which the ultrasound beam is directed. The image generation unit 18 converts the ultrasound image data into video signals, and outputs the video signals to the display 34. The display 34 displays an ultrasound image based on the ultrasound image data on the basis of the video signals.

The position measurement unit 10 is connected to the controller 20 via a measurement signal line 40. The position sensor 36 is provided in the ultrasound probe 12. A sensor conductive wire 38 is drawn out from the position sensor 36. The sensor conductive wire 38 is bundled with the cable 32 of the ultrasound probe 12 or is included in the cable 32 of the ultrasound probe 12. The sensor conductive wire 38 extends from the position sensor 36 to the ultrasound diagnostic apparatus main body 14 and is connected to the position measurement unit 10 together with the measurement signal line 40.

The position measurement transmitter 42 is connected to the position measurement unit 10. The position measurement unit 10 outputs position measurement signals to the position measurement transmitter 42. The position measurement transmitter 42 transmits magnetic field position measurement signals by a magnetic field on the basis of the position measurement signals.

The position sensor 36 detects the magnetic field position measurement signals and outputs the sensor signals to the position measurement unit 10 via the sensor conductive wire 38 on the basis of the detected magnetic field position measurement signals. The position measurement unit 10 obtains positional information of the position sensor 36 on the basis of the sensor signals. The positional information may include a three-dimensional coordinate value (x, y, z) in three-dimensional xyz coordinates defined by the position measurement unit 10. The position measurement unit 10 outputs the positional information of the position sensor 36 to the controller 20 via the measurement signal line 40.

The controller 20 causes the image generation unit 18 to execute processing of associating the ultrasound image data with the positional information of the position sensor 36. For example, in a case where the ultrasound beam is repeatedly scanned across the subject and the image generation unit 18 sequentially generates the ultrasound image data over time, the positional information may be associated with the ultrasound image data of each frame. For example, in a case where a user generates volume data by transporting the ultrasound probe 12 along a body surface of the subject with the hand, information in which the positional information is associated with the ultrasound image data of each frame may be stored in the memory 24.

The memory 24 may store image data of the subject acquired in advance by another device. The external device image data may be CT scan data or MRI image data. The image generation unit 18 may display, on the display 34, an image based on the external device image data stored in the memory 24 and an image based on the ultrasound image data generated by the image generation unit 18 in a comparable manner. The image generation unit 18 may display the external device image and the ultrasound image on the display 34 such that the external device image is visible through the ultrasound image generated by the image generation unit 18, for example.

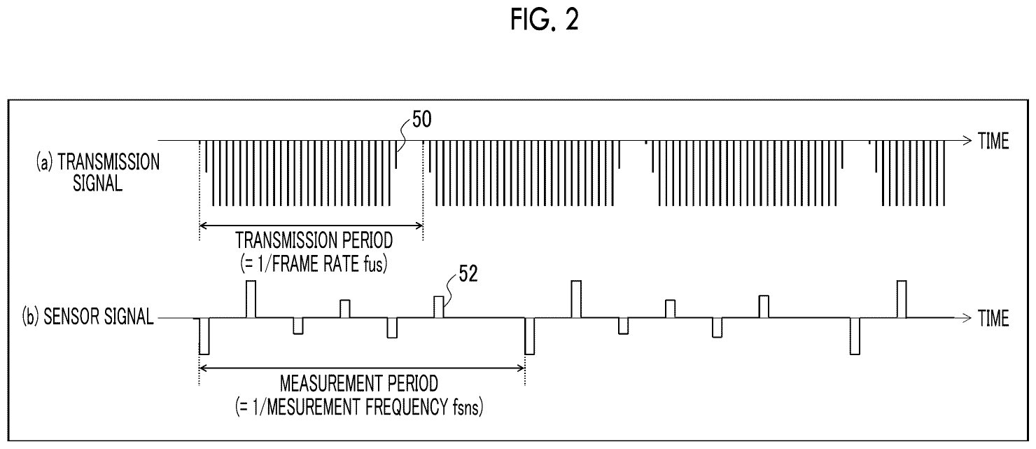

Row (a) of FIG. 2 illustrates a time waveform of the transmission signal output from the ultrasound transmitter and receiver 16 to the ultrasound probe 12. The transmission signal is a signal in which the reciprocal of a frame rate fus is set as a single period. A plurality of transmission pulses 50 are included in a single period (one transmission period) of the transmission signal. A plurality of ultrasound pulses are transmitted from the ultrasound probe 12 during one transmission period.

Row (b) of FIG. 2 illustrates a time waveform of the sensor signal. A plurality of position measurement pulses 52 required per single position measurement are included in a single period (1/fsns) of the sensor signal. In the following description, the reciprocal of the single period of the sensor signal is referred to as a measurement frequency. The position measurement signal output from the position measurement unit 10 to the position measurement transmitter 42 has the same period as the sensor signal, and includes a plurality of pulses required per single position measurement, as in the sensor signal.

The sensor conductive wire 38 drawn from the position sensor 36 is bundled with the cable 32 of the ultrasound probe 12 or is included in the cable 32 of the ultrasound probe 12. Therefore, in a case where the transmission signal is output from the ultrasound transmitter and receiver 16, a noise voltage or a noise current based on the transmission signal may be generated in the sensor conductive wire 38 and interfere with the sensor signal. As a result, an error may occur in the positional information obtained by the position measurement unit 10.

This error may be evaluated on the basis of the variation in the position coordinate values included in the positional information obtained over a plurality of times. The variation in the position coordinate values includes, for example, standard deviation of the position coordinate values obtained over a plurality of times. For example, in a case where the positional information is represented by xyz three-dimensional coordinates, the variation may be represented by the standard deviation of any one coordinate value of an x-axis coordinate value, a y-axis coordinate value, or a z-axis coordinate value. In addition, for example, in a case where the positional information is represented by three-dimensional polar coordinates (a distance r from an origin, an azimuthal angle θ as viewed from the origin, and an elevation angle φ as viewed from the origin), the variation may be represented by the standard deviation of the distance r=(x²+y²+z²)/2. In addition, the variation may be a value obtained by averaging a dispersion of the position coordinate values obtained over a plurality of times or an absolute value of a value obtained by subtracting an average value from each value.

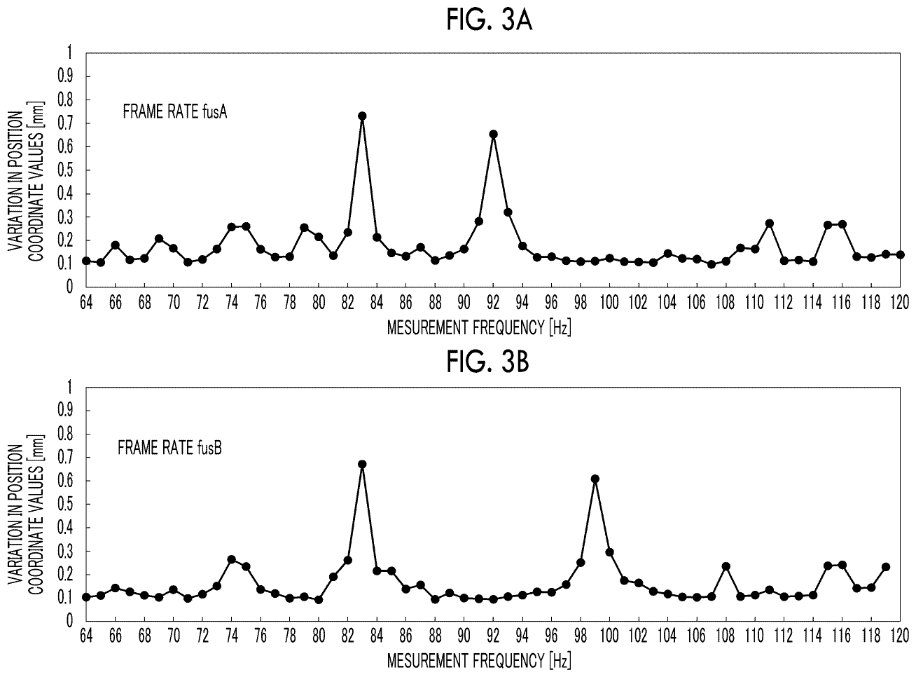

FIGS. 3A and 3B illustrate variation in the position coordinate values with respect to the measurement frequency for two different frame rates fusA and fusB. As is clear from FIGS. 3A and 3B, the variation is maximized at a specific measurement frequency. In a case where the frame rate of the transmission signal is changed, the measurement frequency at which the variation is maximized is changed. In addition, in a case where the frame rate of the transmission signal is changed, an interference-avoidance measurement frequency, which is a frequency at which the variation is equal to or less than a predetermined threshold value, is also changed. Similarly, in a case where the measurement frequency is changed, an interference-avoidance frame rate, which is a frame rate at which the variation is equal to or less than a predetermined threshold value, is also changed.

Therefore, the control database 22 of the ultrasound diagnostic apparatus 100 includes a frame rate table 28. The frame rate table 28 is a table in which the measurement frequency and the interference-avoidance frame rate are associated with each other. The controller 20 refers to the frame rate table 28, and acquires, regarding the measurement frequency in a case where the position measurement transmitter 42 transmits the magnetic field position measurement signal, the interference-avoidance frame rate associated with the measurement frequency in the frame rate table 28. The controller 20 controls the ultrasound transmitter and receiver 16 to set a frame rate of the transmission signal output from the ultrasound transceiver 16 to the ultrasound probe 12, to the interference-avoidance frame rate.

In the frame rate table 28, the interference-avoidance frame rate is associated with each measurement frequency. The controller 20 sets the frame rate of the transmission signal input to the ultrasound probe 12 to the interference-avoidance frame rate associated with the measurement frequency of the position measurement signal, and thereby suppresses the interference received from the transmission signal by the position measurement unit 10 via the sensor conductive wire 38. Accordingly, the accuracy of the positional information acquired by using the position sensor 36 is improved.

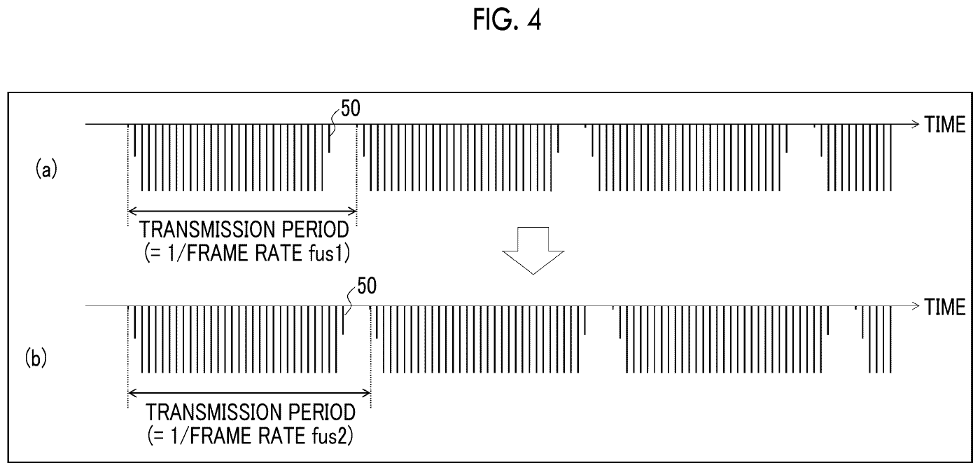

FIG. 4 is a diagram illustrating transmission signals before and after the frame rate is changed. Row (a) of FIG. 4 illustrates transmission signals before the frame rate is decreased. Row (b) of FIG. 4 illustrates transmission signals after the frame rate is decreased. In the transmission signals illustrated in row (b) of FIG. 4, a transmission pulse 50 is added to the transmission signal illustrated in row (a) of FIG. 4 during a single transmission period. That is, the number of transmission pulses 50 per single transmission period of the transmission signals illustrated in row (b) of FIG. 4 is larger than the number of transmission pulses 50 per single transmission period of the transmission signals illustrated in row (a) of FIG. 4. In addition, the frame rate fus2 of the transmission signals illustrated in row (b) of FIG. 4 is smaller than the frame rate fus1 of the transmission signals illustrated in row (a) of FIG. 4.

As compared with the transmission signals of row (b) of FIG. 4, with the transmission signals of row (a) of FIG. 4, the information included in the ultrasound image data of one frame is smaller, but a video with smooth motion is displayed. On the other hand, as compared with the transmission signals of row (a) of FIG. 4, with the transmission signals of row (b) of FIG. 4, a video with a non-smooth motion is displayed, but the amount of information included in the ultrasound image data of one frame is increased.

Here, processing of increasing the number of transmission pulses 50 per single transmission period to decrease the frame rate is illustrated. Instead of such processing, processing of decreasing the number of pulses per single transmission period to increase the frame rate may be executed.

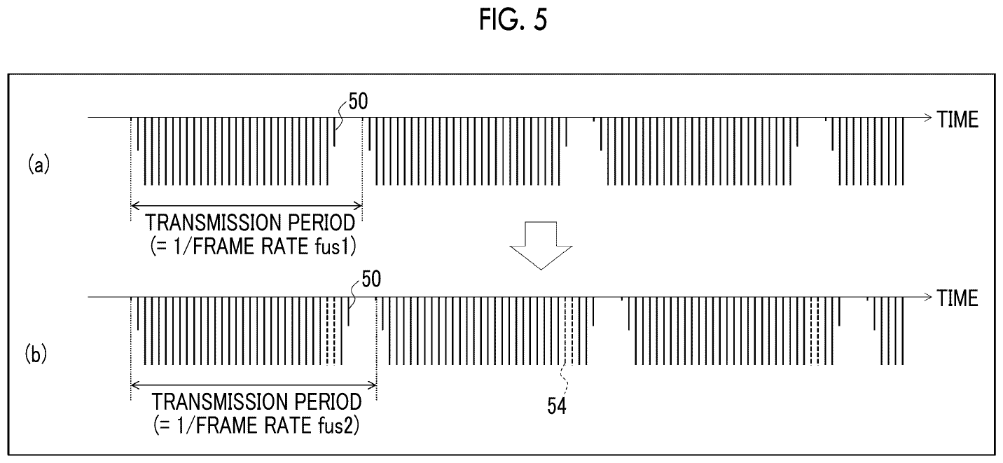

Note that in a case where the transmission pulse 50 is added for the single transmission period, the added transmission pulse 50 may be a dummy pulse that does not contribute to the generation of the ultrasound image data. Row (a) of FIG. 5 illustrates transmission signals before the frame rate is decreased. Row (b) of FIG. 5 illustrates transmission signals after the frame rate is decreased. In row (b) of FIG. 5, a dummy pulse 54 added to increase the number of pulses per single transmission period is indicated by a broken line.

The controller 20 and the ultrasound transmitter and receiver 16 are configured to recognize a position of the dummy pulse 54 on a time axis. The ultrasound transmitter and receiver 16 outputs the phase summation reception signal obtained by excluding the dummy pulse 54 included in the reception signal, to the image generation unit 18. That is, the controller 20 controls the image generation unit 18 such that the dummy pulse 54 does not contribute to the generation of the ultrasound image data. With such processing, the effective frame rate (the frame rate of the video displayed on the display 34) in the image generation unit 18 is maintained while the apparent frame rate is decreased. As a result, it is easy to change the processing executed by the image generation unit 18 on the ultrasound image data of one frame.

The control database 22 in the ultrasound diagnostic apparatus 100 according to the present embodiment includes a measurement frequency table 30. The measurement frequency table 30 is a table in which the frame rate and the interference-avoidance measurement frequency are associated with each other. The controller 20 refers to the measurement frequency table 30, and acquires, regarding the frame rate in a case where the ultrasound transmitter and receiver 16 outputs the transmission signal to the ultrasound probe 12, the interference-avoidance measurement frequency associated with the frame rate in the measurement frequency table 30. The controller 20 controls the position measurement unit 10 to set the measurement frequency of the position measurement signal output to the position measurement transmitter 42 by the position measurement unit 10, to the interference-avoidance measurement frequency.

As illustrated with reference to FIGS. 3A and 3B, there is an interference-avoidance measurement frequency at which the variation in the position coordinate values is equal to or less than a predetermined threshold value for a specific frame rate. In the measurement frequency table 30, the interference-avoidance measurement frequency is associated with each frame rate. The controller 20 sets the measurement frequency of the position measurement signal to the interference-avoidance measurement frequency associated with the frame rate, and thereby suppresses the interference received from the transmission signal by the position measurement unit 10 via the sensor conductive wire 38. Accordingly, the accuracy of the positional information acquired by the position sensor 36 is improved.

The positional information measured by the position measurement unit 10 includes an offset error based on the characteristics of each of the position sensor 36 and the position measurement unit 10. In a case where the positional information is represented by three-dimensional coordinate values (x, y, z), the controller 20 subtracts the offset adjustment value of each position coordinate value from the x-axis coordinate value, the y-axis coordinate value, and the z-axis coordinate value indicated by the positional information, and obtains the positional information in which the offset error is compensated.

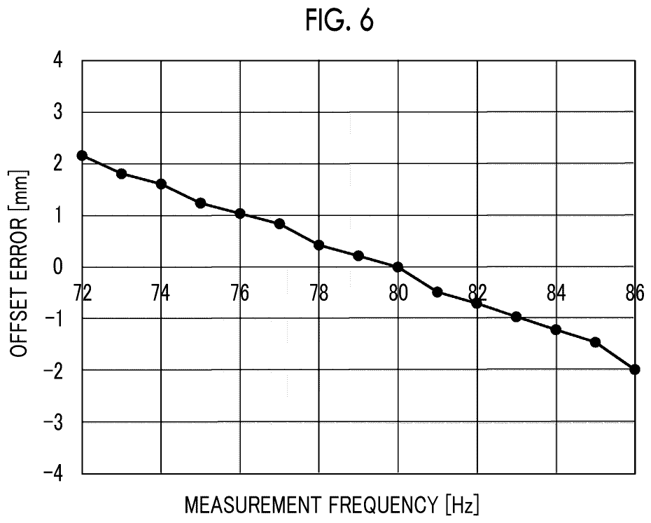

The offset error fluctuates according to a change in the measurement frequency. FIG. 6 illustrates an example of the offset error with respect to the measurement frequency. The lateral axis indicates the measurement frequency, and the vertical axis indicates the offset error for one coordinate axis. In the example illustrated in FIG. 6, the offset error is zero in a case where the measurement frequency is 80 Hz. In a case where the measurement frequency exceeds 80 Hz, the offset error is increased in the negative direction (is decreased), and in a case where the measurement frequency is less than 80 Hz, the offset error is increased.

As described above, since the offset error fluctuates according to the change in the measurement frequency, the controller 20 changes the offset adjustment value for compensating the offset error in a case where the measurement frequency is changed.

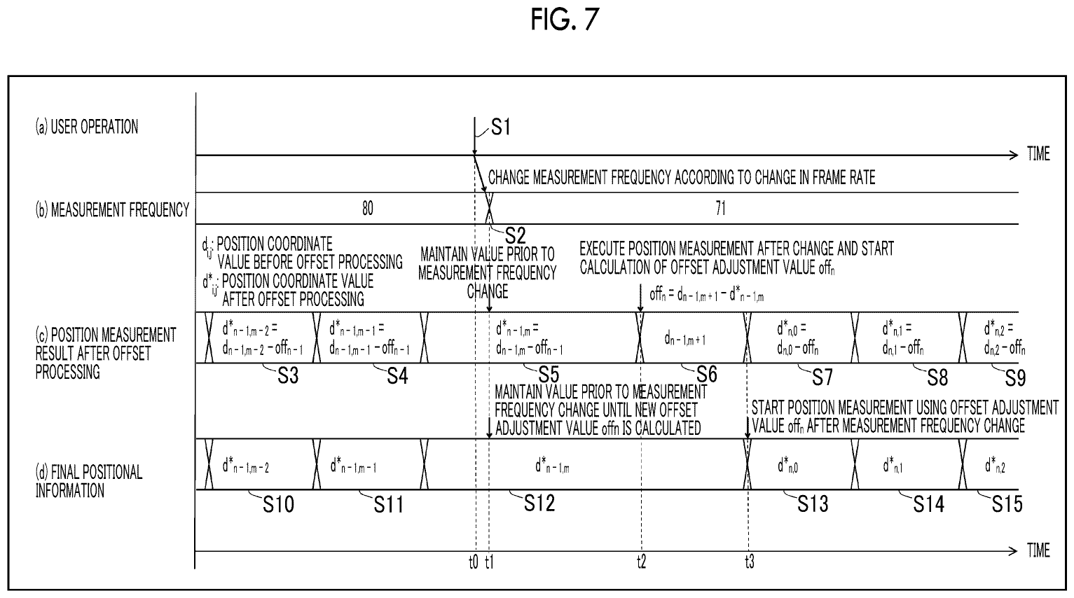

Row (a) to (d) of FIG. 7 illustrate timing charts of processing of changing the offset adjustment value in accordance with the change in the measurement frequency. Row (a) of FIG. 7 illustrates that the user has performed an operation (S1) of changing the measurement frequency at time t=t0. Row (b) of FIG. 7 illustrates that the controller 20 has changed the measurement frequency from 80 Hz to 71 Hz at time t=t1 by the operation of the user (S2).

Row (c) of FIG. 7 illustrates a position measurement result after the offset processing. The position coordinate value before the offset processing is represented by d_{i,j}, and the position coordinate value after the offset processing is represented by d*_{i,j}. Row (d) of FIG. 7 illustrates the position coordinate value d*_{i,j} to be finally obtained as the final positional information. In a case where the positional information is represented by the xyz three-dimensional coordinate values, the position coordinate values illustrated in row (c) of FIG. 7 are any of the x-axis coordinate value, the y-axis coordinate value, and the z-axis coordinate value. In addition, the offset processing refers to processing of subtracting the offset adjustment value of each position coordinate value from the x-axis coordinate value, the y-axis coordinate value, and the z-axis coordinate value indicated by the positional information to correct each position coordinate value. The integer i indicates a value in the i-th offset processing. The integer j indicates a position coordinate value obtained for the j-th instance.

Before the time t=t1 at which the measurement frequency is changed, the controller 20 performs the offset processing using (n−1)-th offset adjustment value offₙ₋₁ on the position coordinate value d_{n-1,m-2} obtained for the (m−2)-th instance to obtain a position coordinate value d*_{n-1,m-2}=d_{n-1,m-2}−offₙ₋₁ after the offset processing (S3). The controller 20 reflects the position coordinate value d*_{n-1,m-2} after the offset processing in the final positional information (S10).

Subsequently, the controller 20 performs the offset processing using the (n−1)-th offset adjustment value offₙ₋₁ on the position coordinate value d_{n-1,m-1} obtained for the (m−1)-th instance to obtain a position coordinate value d*_{n-1,m-1}=d_{n-1,m-1}−offₙ₋₁ after the offset processing (S4). The controller 20 reflects the position coordinate value d*_{n-1,m-1} after the offset processing in the final positional information (S11).

The controller 20 performs the offset processing using (n−1)-th offset adjustment value offₙ₋₁ on the position coordinate value d_{n-1,m} obtained for the m-th instance to obtain a position coordinate value d*_{n-1,m}=d_{n-1,m}−offₙ₋₁ after the offset processing (S5). While this processing is being executed, the measurement frequency is changed at time t=t1, and the controller 20 starts processing of newly obtaining an n-th offset adjustment value offₙ at time t=t2 (S6). The controller 20 maintains a state in which the position coordinate value d*_{n-1,m} after the (n−1)-th offset processing is reflected in the final positional information until time t=t3 at which the n-th offset adjustment value offₙ is obtained (S12).

The controller 20 newly obtains an n-th offset adjustment value offₙ=d_{n-1,m+1}−d*_{n-1,m} by subtracting the position coordinate value d*_{n-1,m} after the offset processing, which is previously obtained, from the position coordinate value d_{n-1,m+1} obtained for the (m+1)-th instance (S6).

With the completion of the processing of obtaining the n-th offset adjustment value offₙ at time t=t3, the controller 20 resets the value of m to zero, performs the offset processing using the n-th offset adjustment value offₙ on the position coordinate value d_{n,0} obtained in the 0-th instance, and obtains a position coordinate value d*_{n,0}=d_{n,0}−offₙ after the offset processing (S7). The controller 20 reflects the position coordinate value d*_{n,0} after the offset processing in the final positional information (S13).

Subsequently, the controller 20 performs the offset processing using the n-th offset adjustment value offₙ on the position coordinate value d_{n,1} obtained in the first instance, and obtains a position coordinate value d*_{n,1}=d_{n,1}-offₙ after the offset processing (S8). The controller 20 reflects the position coordinate value d*_{n,1} after the offset processing in the final positional information (S14). The controller 20 further performs the offset processing using the n-th offset adjustment value offₙ on the position coordinate value d_{n,2} obtained in the second instance, and obtains a position coordinate value d*_{n,2}=d_{n,2}−off₁ after the offset processing (S9). The controller 20 reflects the position coordinate value d*_{n,2} after the offset processing in the final positional information (S15).

As described above, the controller 20 corrects the position coordinate value (position measurement value) obtained by the sensor signal, on the basis of the offset adjustment value obtained in advance (S3, S10, S4, S11). In a case where the measurement frequency is changed (S1, S2), the offset adjustment value offₙ is updated on the basis of a difference between the corrected position measurement value (d*_{n-1,m}) obtained before the measurement frequency is changed and the position measurement value (d_{n-1,m+i}) before correction obtained after the measurement frequency is changed (S6). The controller 20 corrects the position measurement value (d_{n, 0}) before correction obtained after the measurement frequency is changed, on the basis of the updated offset adjustment value (d*_{n, 0}=d_{n, 0}−offₙ) (S7). The controller 20 reflects the position coordinate value d*_{n,0} after the offset processing in the final positional information (S13).

With such processing, the fluctuation of the offset error generated in the positional information due to the change in the measurement frequency is compensated, and the accurate positional information of the position sensor 36 is acquired by the controller 20.

In the above, first processing of setting the frame rate on the basis of the measurement frequency and the frame rate table 28 and second processing of setting the measurement frequency on the basis of the frame rate and the measurement frequency table 30 have been described. Only one of the first processing and the second processing may be performed, or both the first processing and the second processing may be performed. That is, processing of setting at least one of the frame rate or the measurement frequency may be executed on the basis of the control database 22.

In the above-described embodiment, a sensor that detects a magnetic field is used as the position sensor 36. In addition to the sensor that detects a magnetic field, a position sensor that detects an electromagnetic field (radio wave), light, an ultrasonic wave, or the like may be used. In this case, a transmitter that transmits an electromagnetic field, light, an ultrasonic wave, or the like is used instead of the position measurement transmitter 42.

### Configuration of Present Disclosure

Configuration 1: An ultrasound diagnostic apparatus including an ultrasound probe, and a position sensor that is provided in the ultrasound probe, the ultrasound diagnostic apparatus including an information processing unit configured to generate ultrasound image data on the basis of ultrasound waves received by the ultrasound probe and measure a position of the ultrasound probe on the basis of a sensor signal output from the position sensor; and a control database in which a frame rate in a case where the ultrasound image data is generated and a measurement frequency of the sensor signal are associated with each other, in which the information processing unit sets at least one of the frame rate or the measurement frequency on the basis of the control database.

Configuration 2: In the ultrasound diagnostic apparatus according to Configuration 1, the control database includes a measurement frequency table in which the frame rate and an interference-avoidance measurement frequency are associated with each other, and the information processing unit sets the measurement frequency to the interference-avoidance measurement frequency associated with the frame rate in the measurement frequency table.

Configuration 3: In the ultrasound diagnostic apparatus according to Configuration 1 or 2, the information processing unit corrects a position measurement value obtained by the sensor signal, on the basis of an offset adjustment value obtained in advance, updates, in a case where the measurement frequency is changed, the offset adjustment value on the basis of a difference between the corrected position measurement value obtained before the measurement frequency is changed and the position measurement value before correction obtained after the measurement frequency is changed, and corrects the position measurement value before correction obtained after the measurement frequency is changed, on the basis of the offset adjustment value before the update.

Configuration 4: In the ultrasound diagnostic apparatus according to any one of Configurations 1 to 3, the control database includes a frame rate table in which the measurement frequency and an interference-avoidance frame rate are associated with each other, and the information processing unit sets the frame rate to the interference-avoidance frame rate associated with the measurement frequency in the frame rate table.

Configuration 5: In the ultrasound diagnostic apparatus according to any one of Configurations 1 to 4, the information processing unit changes the number of ultrasound pulses transmitted by the ultrasound probe per single transmission period to change the frame rate.

Configuration 6: In the ultrasound diagnostic apparatus according to Configuration 5, the information processing unit causes, among a plurality of ultrasound pulses transmitted by the ultrasound probe per single transmission period, the ultrasound pulses increased compared to before the frame rate is changed not to contribute to generation of the ultrasound image data.

What is claimed is:

1. An ultrasound diagnostic apparatus including an ultrasound probe, and a position sensor that is provided in the ultrasound probe, the ultrasound diagnostic apparatus comprising:
an information processing unit configured to generate ultrasound image data on the basis of ultrasound waves received by the ultrasound probe and measure a position of the ultrasound probe on the basis of a sensor signal output from the position sensor; and
a control database in which a frame rate in a case where the ultrasound image data is generated and a measurement frequency of the sensor signal are associated with each other,
wherein the information processing unit sets at least one of the frame rate or the measurement frequency on the basis of the control database.

2. The ultrasound diagnostic apparatus according to claim 1,
wherein the control database includes a measurement frequency table in which the frame rate and an interference-avoidance measurement frequency are associated with each other, and
the information processing unit sets the measurement frequency to the interference-avoidance measurement frequency associated with the frame rate in the measurement frequency table.

3. The ultrasound diagnostic apparatus according to claim 1,
wherein the information processing unit
corrects a position measurement value obtained by the sensor signal, on the basis of an offset adjustment value obtained in advance,
updates, in a case where the measurement frequency is changed, the offset adjustment value on the basis of a difference between the corrected position measurement value obtained before the measurement frequency is changed and the position measurement value before correction obtained after the measurement frequency is changed, and
corrects the position measurement value before correction obtained after the measurement frequency is changed, on the basis of the offset adjustment value before the update.

4. The ultrasound diagnostic apparatus according to claim 2,
wherein the information processing unit
corrects a position measurement value obtained by the sensor signal, on the basis of an offset adjustment value obtained in advance,
updates, in a case where the measurement frequency is changed, the offset adjustment value on the basis of a difference between the corrected position measurement value obtained before the measurement frequency is changed and the position measurement value before correction obtained after the measurement frequency is changed, and
corrects the position measurement value before correction obtained after the measurement frequency is changed, on the basis of the offset adjustment value before the update.

5. The ultrasound diagnostic apparatus according to claim 1,
wherein the control database includes a frame rate table in which the measurement frequency and an interference-avoidance frame rate are associated with each other, and
the information processing unit sets the frame rate to the interference-avoidance frame rate associated with the measurement frequency in the frame rate table.

6. The ultrasound diagnostic apparatus according to claim 1,
wherein the information processing unit changes the number of ultrasound pulses transmitted by the ultrasound probe per single transmission period to change the frame rate.

7. The ultrasound diagnostic apparatus according to claim 5,
wherein the information processing unit changes the number of ultrasound pulses transmitted by the ultrasound probe per single transmission period to change the frame rate.

8. The ultrasound diagnostic apparatus according to claim 5,
wherein the information processing unit causes, among a plurality of ultrasound pulses transmitted by the ultrasound probe per single transmission period, the ultrasound pulses increased compared to before the frame rate is changed not to contribute to generation of the ultrasound image data.
